(19) United States
(12) Patent Application Publication
(10) Pub. No.: US 2004/0115319 A1
(43) Pub. Date: Jun. 17, 2004

(54) Food-borne pathogen and spoilage detection device and method

(75) Inventors: Roger Morris, Sebastian, FL (US); John A. McMorris III, Indialantic, FL (US); Galo Acosta, Sebastian, FL (US); Jerry Hill, Cocoa, FL (US); Alan R. Tank, Bethesda, MD (US); Kyle Newman, Lexington, KY (US); Alan Bishop, Sebastian, FL (US)

Correspondence Address: Allen, Dyer, Doppelt, Millbrath & Gilchrist, P.A., Suite 1401, 255 South Orange Avenue, Orlando, FL, 32801, US

(73) Assignee: AgCert International, LLC, Sebastian, FL

(21) Appl. No.: 10/659,222

(22) Filed: Sep. 10, 2003

Related U.S. Application Data
(60) Provisional application No. 60/411,068, filed on Sep. 16, 2002.
(60) Provisional application No. 60/421,699, filed on Oct. 28, 2002.
(60) Provisional application No. 60/484,869, filed on Jul. 3, 2003.

(51) Int. Cl. G01N 33/02

(52) U.S. Cl. 426/231

(57) ABSTRACT

A device for detecting bacteria in a perishable food product includes a gas-permeable sensor housing positionable within an interior of food packaging. A pH indicator is positioned within the housing for detecting a change in a gaseous bacterial metabolite concentration that is indicative of bacterial growth, wherein a pH change is effected by a presence of the metabolite. The housing and the pH indicator are preferably safe for human consumption. A method for detecting bacteria in a perishable food product includes supporting a food product by a food packaging element and positioning a gas-permeable sensor housing within an interior of the food packaging element, the sensor including a pH indicator. The food product and the housing are sealed within the food packaging, and the pH indicator is monitored for a bacterial concentration in the food product in excess of a predetermined level.

### CROSS-REFERENCE TO RELATED APPLICATIONS

This application claims priority to provisional applications Serial No. 60/411,068, filed Sep. 16, 2002, entitled “Food Borne Pathogen Detection Device and Method for Packaged Meat”; Serial No. 60/421,699, filed Oct. 28, 2002, entitled “Food Borne Pathogen Detection Device and Method for Packaged Perishable Foods”; and Serial No. 60/484,869, filed Jul. 3, 2003, entitled “Food Borne Pathogen Detection Device and Method.”

### FIELD OF THE INVENTION

The present invention generally relates to pathogen detection devices and methods, and, in particular, to devices and methods for detecting food-borne pathogens and spoilage.

### BACKGROUND OF THE INVENTION

Food-borne diseases as well as food spoilage remain a significant burden in the global food supply. In the U.S. alone there are 76 million cases of food-borne illnesses annually, which is equivalent to one in every four Americans, leading to approximately 325,000 hospitalizations and over 5000 deaths annually.

According to the GAO and USDA, food-borne pathogens cause economic losses ranging from $7 billion to $37 billion dollars in health care and productivity losses. Hazard Analysis and Critical Control Point (HACCP) regulations state that a hazard analysis on a food product must include food-safety analyses that occur before, during, and after entry into an establishment. There is a clear need to ensure that food transported from the processor to the consumer is as safe as possible prior to consumption. For example, the development of antibiotic resistance in food-borne pathogens, the presence of potential toxins, and the use of growth hormones all indicate a need for further development of HACCP procedures to ensure that safer food products are delivered to the consumer.

Meat, for example, is sampled randomly at the processor for food-borne pathogens. Generally, no further testing occurs before the meat is consumed, leaving the possibility of unacceptable levels of undetected food-borne pathogens, such as Salmonella spp. and Listeria spp., as well as spoilage bacteria, such as Pseudomonas spp. and Micrococcus spp. being able to multiply to an undesirable level during the packaging, transportation, and display of the product. Subsequently the food product is purchased by the consumer and is transported and stored in uncontrolled conditions that only serve to exacerbate the situation, all these events occurring prior to consumption.

Retailers generally estimate shelf life and thus freshness with a date stamp. This method is inaccurate for two key reasons: First, the actual number of bacteria on the meat at the processor is unknown, and second, the actual time-temperature environment of the package during its shipment to the retailer is unknown. As an example, a temperature increase of less than 3° C. can shorten food shelf life by 50% and cause a significant increase in bacterial growth over time. Indeed, spoilage of food may occur in as little as several hours at 37° C. based on the universally accepted value of a total pathogenic and non-pathogenic bacterial load equal to 1×10⁷ cfu/gram or less on food products. This level has been identified by food safety opinion leaders as the maximum acceptable threshold for meat products.

While many shelf-life-sensitive food products are typically processed and packaged at a central location, this has not been true in the meat industry. The recent advent of centralized case-ready packaging as well as cryovac packaging for meat products offers an opportunity for the large-scale incorporation of sensors that detect both freshness and the presence of bacteria.

A number of devices are known that have attempted to provide a diagnostic test that reflects either bacterial load or food freshness, including time-temperature indicator devices. To date none of these devices has been widely accepted either in the consumer or retail marketplace, for reasons that are specific to the technology being applied. First, time-temperature devices only provide information about integrated temperature history, not about bacterial growth; thus it is possible, through other means of contamination, to have a high bacterial load on food even though the temperature has been maintained correctly. Wrapping film devices require actual contact with the bacteria; if the bacteria are internal to the exterior food surface, then an internally high bacterial load on the food does not activate the sensor. Ammonia sensors typically detect protein breakdown and not carbohydrate breakdown. Since bacteria initially utilize carbohydrates, these sensors have a low sensitivity in most good applications, with the exception of seafood.

Therefore, it would be desirable to provide a device, food packaging, and associated methods for detecting at least a presence of bacteria in a perishable food product.

### SUMMARY OF THE INVENTION

The present invention, a first aspect of which includes a device for detecting a presence of bacteria in a perishable food product, comprises a gas-permeable sensor housing that is positionable in an interior of food packaging. The device further includes a pH indicator that is positioned within the housing. The indicator is for detecting a change in a gaseous bacterial metabolite concentration that is indicative of bacterial growth, wherein a pH change is effected by a presence of the metabolite. In a particular embodiment, the housing and the pH indicator are safe for human consumption.

Another aspect of the invention includes a method for detecting a presence of bacteria in a perishable food product. This method comprises the steps of supporting a food product by a food packaging element and positioning a gas-permeable sensor housing within an interior side of the food packaging element. The sensor comprises a pH indicator that is adapted to detect a change in a gaseous bacterial metabolite concentration that is indicative of bacterial growth. A pH change is effected by a presence of the metabolite. The food product and the housing are sealed within the food packaging, and the pH indicator is monitored for a bacterial concentration in the food product in excess of a predetermined level.

A further aspect of the invention includes a method of packaging a perishable food product. This method comprises the steps of supporting a food product by a food packaging element and positioning a gas-permeable sensor housing as above. The food product and the housing are then sealed within the food packaging.

An additional aspect of the invention includes a method of making a device for detecting a presence of bacteria in a perishable food product. This method comprises the steps of positioning a pH indicator within a gas-permeable sensor housing as above, the housing positionable in an interior of food packaging.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIGS. 1A-C illustrate the time evolution of bacterial growth detection, with a sensor packaged with a perishable food item (FIG. 1A), growth of bacterial colonies on the food, the bacteria emitting a gaseous metabolite (FIG. 1B), and an observable change exhibited by the sensor in response to a decrease in pH (FIG. 1C).

FIG. 2A is a top, side perspective view of a first embodiment of a bacterial growth detector.

FIG. 2B is a top, side perspective view of a second embodiment of a bacterial growth detector.

FIG. 2C is a top, side perspective view of a third embodiment of a bacterial growth detector.

FIG. 2D is a top, side perspective view of a fourth embodiment of a bacterial growth detector.

FIG. 2E is a top, side perspective view of a fifth embodiment of a bacterial growth detector.

FIG. 2F is a top, side perspective view of a sixth embodiment of a bacterial growth detector.

FIG. 2G is a top, side perspective view of a seventh embodiment of a bacterial growth detector.

FIG. 3A illustrates an integrated time-temperature indicator of food freshness.

FIG. 3B illustrates a combined time-temperature and pH indicator for determining both food freshness and bacterial growth in a unitary device.

### DETAILED DESCRIPTION OF THE PREFERRED EMBODIMENTS

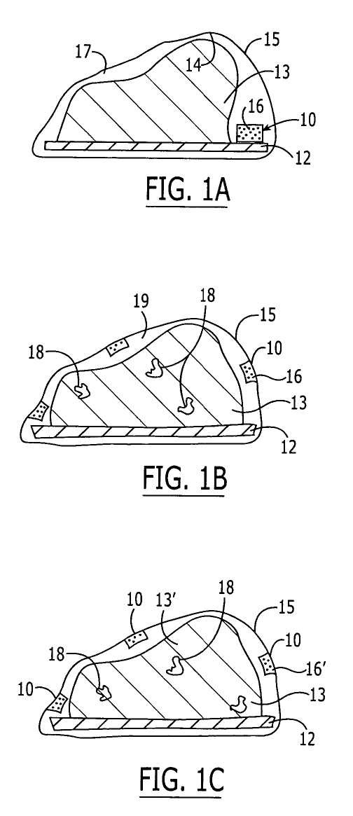

A description of multiple embodiments of the present invention will now be presented with reference to FIGS. 1A-3B.

The present device addresses the need for a device, food packaging, and associated methods for detecting at least a presence of bacteria in a perishable food product. The embodiments of the device provide a quantitative measure of bacterial load and detect the presence of bacteria in or on the food product. In addition, in a particular embodiment, the device comprises a composition that may be consumed safely if mistakenly eaten. A time-temperature device may also be included in certain embodiments to provide additional information along the food supply chain on any departure from recommended temperature maintenance. Consumer-packaged (cooked or uncooked) foods may also be stored in containers (such as sealable bags or plastic containers) with both bacterial and/or time-temperature sensors providing the consumer with a measure of food freshness and safety.

In the following description it is to be understood that the sensor embodiments provide a change that may be based on absorbance (transmittance), fluorescence, or luminescence, the change being observable visually and/or using an optical instrument. Additionally, the sensor or indicator described may be chemically or physically attached to a solid support. For example, the sensor may be positioned within the food package carried by the packaging elements such as the wrapper or the tray that carries the food products. Alternatively, the sensor may simply be placed within the package resting on either the food product or on the package itself. Indeed, since carbon dioxide is heavier than air, it is sometimes preferable that the sensor be located near a deep part of the container.

The device and methods are adapted to detect the presence of bacteria in shelf-life-sensitive packagable food products such as meats, poultry, fish, seafood, fruits, and vegetables using an on-board device comprising an indicator housing and a sensor (or sensors) located within the housing. The device is incorporated within a food package along with the food product, which is sealed to a substantially gas-tight level. In certain embodiments, it is believed advantageous to isolate the device from direct contact with the food product, and/or to detect the freshness of such packagable foods using a separate or incorporated sensor placed within the food packaging.

A device comprising an aqueous pH indicator, constructed to have an initial, pre-exposure pH opposite to an expected pH shift, is preferably isolated chemically or physically from the typically acidic environment present in a food sample, but unprotected from neutral gases. As bacteria multiply, metabolites are produced and diffuse into the pH indicator. The metabolite is sensed as a pH shift in the indicator, with a pH drop if the indicator is adapted to detect an acid, and a pH increase if the indicator is adapted to detect an alkaline substance.

An exemplary indicator comprises a material adapted to undergo a color change with a change in pH, such as bromothymol blue, phenol red, or cresol red, although these are not intended to be limiting. An edible or nontoxic pH indicator may also be used, such as, but not intended to be limited to, extracts of red cabbage, turmeric, grape, or black carrot, obtained from a natural source such as a fruit or vegetable. Experiments have indicated that a sensor based on a pH indicator is capable of detecting a total pathogenic and non-pathogenic bacterial load equal to 1×10⁷ cfu/gram or less on food products, a level that has been identified by food safety opinion leaders as the maximum acceptable threshold for most food, for example.

In some of the embodiments of the present invention, carbon dioxide is used as a generic indicator of bacterial growth and to quantitatively estimate the level of bacterial contamination present in a sample. As is well known, when carbon dioxide comes into contact with an aqueous solution, the pH drops owing to the formation of carbonic acid, thus making pH an indicator of carbon dioxide concentration and, hence, of bacterial load. All the present embodiments are capable of detecting a total pathogenic and non-pathogenic bacterial load at a level of at least 10⁷ cfu/g.

Another type of pH indicator measures the concentration of another metabolite comprising a volatile organic compound such as ammonia. In this embodiment the sensor comprises an aqueous solution having an initial pH in the acid range, for example, pH 4, effected by the addition of an acid such as hydrochloric acid. As alkaline gases such as ammonia diffuse into the sensor, ammonia reacts with water to form ammonium hydroxide, which in turn raises the pH of the solution. As the pH level rises, a commensurate indicator change occurs, which, when detectable, is representative of food contamination.

A non-pH indicator may also be envisioned, wherein a bacterial metabolite diffuses into a sensor. This embodiment of the sensor comprises a chemical that precipitates out of solution in the presence of the metabolite. As an example, a calcium hydroxide sensor, in a concentration range of 0.0001-0.1M, would form an observable precipitate of calcium carbonate in the presence of sufficient carbon dioxide.

In some embodiments it may be desirable to incorporate a radiation shield into the sensor, to minimize photodegradation of the indicator. For example, a colored dye could be incorporated to attenuate ultraviolet radiation, although this is not intended as a limitation.

A potential disadvantage of some gas sensors based upon sensing pH levels may include the possibility that, once the sensor is exposed to air, or if a pH change occurs within the food packaging, the sensor color could in principle revert to a state wherein the food was indicated as being “safe,” even though a potentially unsafe bacterial load had been indicated previously. Thus it may be desirable in certain instances to incorporate a sensor the changed state of which is nonreversible.

Such a difficulty could be overcome by using a sensor material that is unstable over a time period commensurate with a time over which the sensor is desired to operate. For example, anthrocyanine-based pH indicators derived from vegetables can break down via oxidation over a period spanning hours or days, which make their indication substantially irreversible. Alternatively, a precipitating embodiment could be used, either alone or in combination with one or more other sensors, wherein the precipitate does not dissipate, providing a substantially irreversible indicator.

A plurality of shapes and configurations of such a sensor may be appreciated by one of skill in the art, including, but not limited to, disc-like, spherical, or rectangular. Disc-shaped elements are shown herein for several of the examples, since it is believed advantageous to provide as much surface area as possible for enhancing gas diffusion into the sensor, to minimize state-changing time, and, therefore, to optimize sensitivity.

The general operation of the device is illustrated in FIGS. 1A-1C, wherein a detector device is provided that comprises a gas-permeable sensor 10. The sensor 10 comprises an indicator that is adapted to detect a change in a gaseous bacterial metabolite concentration indicative of bacterial growth. A change is effected by a presence of the metabolite, and an observable change in the indicator is commensurate with a concentration of the metabolite.

The sensor 10 is sealed within a food packaging element, here, a tray 12 that is supporting a food product 13. In this embodiment a unitary sensor 10 is positioned within an interior 14 of a sealing film 15 (FIG. 1A). It will be understood by one of skill in the art that a plurality of sensors 10 could be used in some cases, and that the packaging element could also comprise, for example, a consumer-type sealable bag or container. An initial state of the sensor 10 is represented by dotted shading 16, the sensor 10 initially sensing a metabolite concentration of the air 17 trapped within the packaging 12,15.

With elapsed time and possible changes in storage temperature, bacterial colonies 18 begin to form on and in the food product 13, the bacterial colonies emitting a gaseous metabolite 19 that diffuses to the sensor 10 (FIG. 1B). The sensor 10 undergoes a chemical change indicative of the concentration of the metabolite 19. When the chemical change is sufficient to cause a detectable change, indicated by hatched shading 16′, a potential spoilage of the food product 13′ is indicated (FIG. 1C). These parameters are dependent upon the characteristics of the sensor 10, each sensor 10 calibrated so that a predetermined metabolite concentration limit is detectable.

Various examples of the device for detecting bacterial contamination of a perishable food product will now be presented.

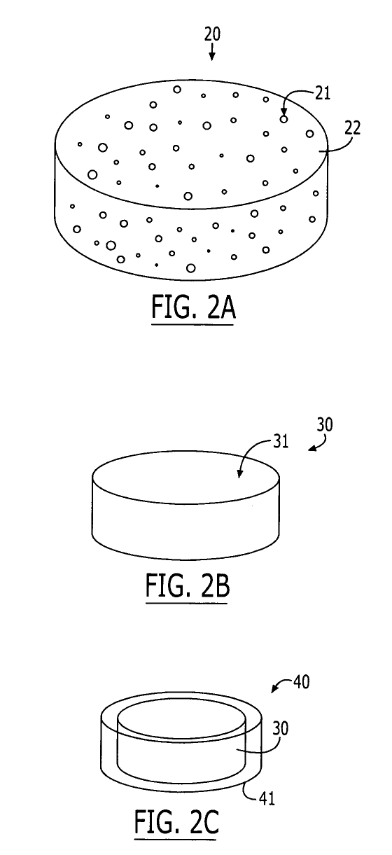

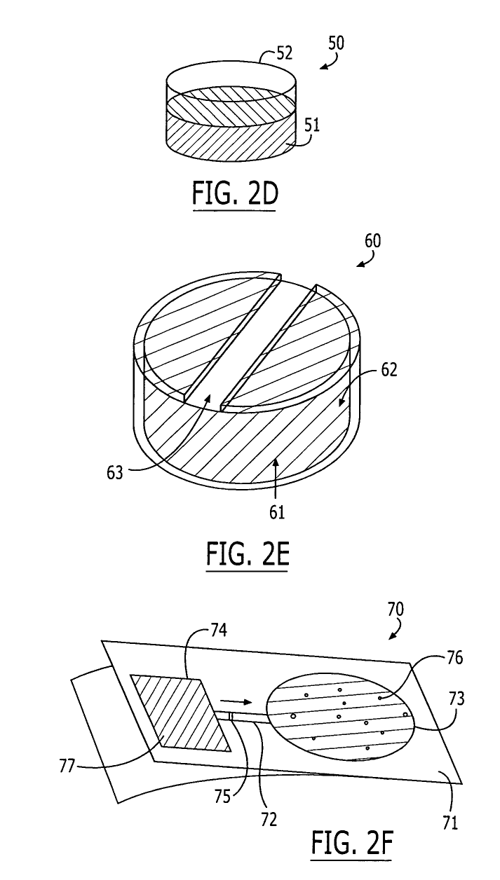

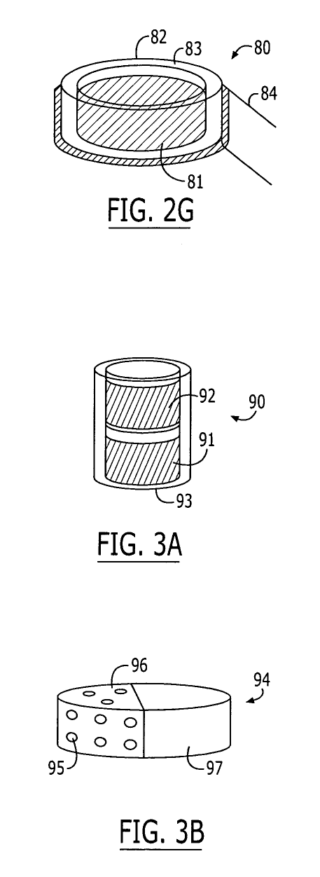

One example of a sensor device 20 (FIG. 2A) may comprise an aqueous pH indicator 21 encapsulated within a silicone housing 22. Silicone is substantially transparent, and is permeable to neutral gases but substantially impermeable to ions such as H⁺. When a metabolite such as carbon dioxide diffuses into the housing 22 and goes into solution in the indicator 21, the resulting pH change is reflected in an observable change, such as a color change, in the indicator 21.

An exemplary form of the sensor device 20 comprises a thin disk, approximately 2.5 cm in diameter and 2-3 mm thick.

Another example of a sensor device 30 (FIG. 2B) may comprise an agar support 31 through which the indicator is substantially uniformly distributed. To form this device 30, the aqueous indicator is mixed into the agar and allowed to cure. Agar is believed advantageous because it is edible and is therefore safe for consumption.

A further example of a sensor device 40 (FIG. 2C) may comprise an agar sensor as described above that has been coated or covered with a proton-impermeable material 41 such as, for example, silicone, or a thin gas-permeable film. Such a coating provides a barrier against charged particles but permits neutral gas entry.

This device 40 could be easily employed, for example, for home use in sealable containers.

Another example of a sensor device 50 (FIG. 2D) may comprise an indicator in solution 51 housed within a gas-permeable, but charged-particle-impermeable, clear housing 52, such as a film or container. A support 53, such as a plastic or cardboard support, may surround a portion of the container 52.

Yet a further example of a sensor device 60 may comprise a housing 61, a reference medium 62, and an indicator medium 63 positioned adjacent the reference medium 62. The reference medium 62 has a substantially constant state, e.g., a substantially immutable color that matches an initial state/color of the indicator medium 63. Thus when the indicator 63 experiences a change of state, the change will be evident from a comparison against the reference 62.

In a particular example (FIG. 2E), the relative positioning of the indicator 63 and reference 62 achieves the formation of an icon indicative of spoilage, for example, a universal stop sign or other warning. In order to achieve such a relative positioning, the indicator medium 63 and the reference medium 62 comprise a unitary material, and the housing 61 comprises a gas barrier such as transparent plastic positioned so as to leave available the indicator area 63 to gas diffusion. Thus only the indicator area 63 changes color under bacterial metabolite production, since the reference area 62 is shielded therefrom.

A further example of a sensor device 70 may comprise a container support 71 and a fluid tube 72 affixed to the support 71. The gas-permeable sensor housing, which is positionable within an interior of food packaging, may comprise a first container 73 and a second container 74 fluidically isolated therefrom. In the example depicted in FIG. 2F, these containers 73,74 comprise “blisters” affixed to a substantially planar base 71 made, for example, of silicone or plastic, at least one of the blisters 73,74 being nonrigid. The fluid tube 72 extends between the blisters 73,74, but a frangible barrier 75 is positioned to block fluid access through the tube 72 unless and until a breaking of the frangible barrier 75 establishes fluid communication between the first 73 and the second 74 blister.

A pH indicator 76 in a substantially desiccated state is positioned within the first blister 73. In a hydrated state, the pH indicator 76 is adapted to detect a change in a gaseous bacterial metabolite concentration indicative of bacterial growth. Alternatively, the pH indicator may be kept in an aqueous acidic state (e.g., pH 3).

A hydrating/alkaline solution 77 is positioned within the second blister 74. The hydrating/alkaline solution 77 preferably has sufficient alkalinity (e.g., pH 10) that a mixture of the pH indicator 76 therewith results in an aqueous pH indicator having an initial pH in the alkaline range.

Thus, in storage, the first 73 and the second 74 blisters are fluidically isolated from each other, and, in use, the pressure is applied to either of the blisters 73,74 to break the barrier 75, permitting the hydrating/alkaline solution 77 to mix with the pH indicator 76, and enabling the pH indicator 76 to perform its intended function.

An advantage of retaining the pH indicator 76 in a desiccated or acidic state is increased shelf life, since some indicators, such as natural pH indicators, tend to be unstable under light exposure, oxidation, and extremes of temperature.

An additional example of a sensor device 80 (FIG. 2G) may comprise an aqueous solution 81 of indicator in silicone or agar, as in the first two examples described above, housed within a gas-permeable, but charged-particle-impermeable, clear housing 82, such as a film or container. The indicator solution 81 is prepared at an alkaline pH, for example, pH 10, using, for example, sodium hydroxide. The container 82 is saturated with carbon dioxide 83, which lowers the pH, increasing the stability of the indicator solution 81.

Activation is achieved by opening the housing 82, such as by using a pull tab 84. Exposure to air permits the carbon dioxide to escape, raising the pH of the indicator solution 81 back to approximately the initial pH, where the device 80 functions most effectively.

Another embodiment of a device 90 may comprise, in addition to a bacterial metabolite sensor 91 as discussed above, a time-temperature integrative sensor 92 (FIG. 3A) that tracks freshness, integrating temperature variations over time. Such a sensor may also be incorporated into the device 70 of FIG. 2F. This device 90 comprises a gas-permeable sensor housing 93 that is positionable within an interior of food packaging. Such a time-temperature integrative sensor 92 provides an integrated temperature history experienced by the food packaging.

For many enzymes to function optimally, a moderate pH, an aqueous environment, and a temperature of approximately 37° C. is preferred. For every 10° C. reduction in temperature, enzyme activity is reduced by a factor of two. Additionally, enzymes tend to be relatively stable at 4° C.

In an embodiment the time-temperature sensor 92 comprises a substrate in solution that may be turned over by an enzyme to produce a color change. At 4° C. very little enzyme activity would occur, resulting in very little color change over the short term. However, at elevated temperatures enzyme activity would significantly increase, resulting in a substantial color change. Such a device would provide an integrated measurement of elevated time/temperature variations that would indicate a higher risk of food spoilage. The rate of reaction may be modified by careful selection of the appropriate enzyme temperature/activity profile. For example, an enzyme such as glucose oxidase may be used to catalyze glucose oxidation to form gluconic acid and hydrogen peroxide, and will, in the presence of an appropriate indicator, produce a color change. Hydrogen peroxide is a strong oxidizing agent that can be used to oxidize chromogenic indicators such as dianisidine producing a colorless to brown color change.

The response of the sensor to the degree of freshness may be adjusted by varying the chemical and/or physical components of the device 90. This in turn permits the tuning of the sensor to the requirements of a particular usage.

Another exemplary time-temperature sensor 92, positioned within a gas-permeable membrane 93, relies on the formation of an acid or carbon dioxide (which subsequently forms carbonic acid in solution).

The detection of bacterial growth and time-temperature integration provides a user with two different pieces of information if the two sensors 91,92 operate independently. In this situation if either sensor 91,92 changes color, for example, the food product would be unacceptable for consumption. These sensors 91,92 may be attached adjacent to each other or stacked.

Both the time-temperature environment and bacterial metabolite production directly and indirectly provide information regarding the freshness, quality, and safety of a perishable food product. Until the present invention a method of combining both indicators into a single, additive sensor has not been available. By combining both indicators into a single sensor 94, an overall estimate of freshness, quality, and safety for any given food product can be provided (FIG. 3B). Both indicators, which should act by experiencing pH changes in the same direction, contribute to form a more sensitive and accurate sensor.

In this example a cocktail is prepared that consists of the bacterial carbon dioxide sensor components and the enzyme/substrate (time-temperature integrator) components combined with a pH indicator in a solution. This cocktail solution 95 is placed in a container 96, comprising, for example, silicone, that is permeable to gases. The container 96 may then be adhered to the inner wall of the transparent film covering the food product, or alternatively placed within the interior space of the packaging. The sensor 94 does not need to be in direct contact with the food, since any carbon dioxide produced by bacteria will permeate the entire container headspace. The carbon dioxide cocktail component consists of a weakly buffered solution. The time-temperature indicator cocktail comprises an enzyme/substrate combination comprising, for example, of a lipase enzyme and an ester substrate. A universal indicator that offers a large spectral change for a relatively small change in pH, e.g., bromothymol blue, is added to the cocktail.

Carbon dioxide produced by bacteria diffuses through the permeable container 96 into the cocktail, forms carbonic acid, and lowers the pH of the solution, resulting in an indicator color change. Depending upon the time-temperature environment, the enzyme turns over the ester substrate, producing fatty acid and alcohol. The fatty acid produced lowers the pH of the solution, also resulting in an indicator color change. Thus the sensor combines the output of both indicators in the same cocktail solution 95 to produce an additive color response.

A reference 97 may also be incorporated in to the sensor design that would indicate that the sensor 94 is functioning according to specifications, and acts as a comparison reference.

If the embodiment of FIG. 2F is utilized, the combined pH indicator and enzyme/substrate components would be desiccated and positioned in the first blister 73, which would be advantageous in the case of unstable pH indicators comprising, for example, natural products.

Experimental Results

The data of Tables 1 and 2 were collected using a silicone sensor prepared as follows: A 5% w/v of bromothymol blue was prepared in aqueous solution. The pH was increased to pH 10 using concentrated sodium hydroxide. Agar was prepared by heating a block of agar to 55° C. 10% v/v of bromothymol blue was added to the agar and the solution was mixed to homogeneity. The agar was poured into 1-in.-diameter transparent containers to a depth of 2 mm and was allowed to cool at room temperature to form a deep blue flexible disk.

Chicken wings obtained from a local grocer were placed in 200-ml plastic sealable containers and incubated at 35 and 4° C. respectively. Agar indicators were prepared and placed adjacent to the chicken wings. The container were then sealed. Drager tubes were used to determine the percent carbon dioxide present when the color changes. At 35° C. an indicator color change was first observed at 2.5 hours and a significant color change at 3 hours, comprising a blue to light green color change. The results provided in Table 1 indicate that approximately 1×10⁷ cfu/g of bacteria were detectable, and could be used as a means for a user to track the freshness and quality of shelf-life-dependent products. The data in Table 2 are provided as a control for chicken wings stored at 4° C.

TABLE 1
Effect of incubation of chicken at 35° C. on biochemical and
microbiological parameters.

| | Carbon Dioxide | Bacterial Concentration |
|---|---|---|
| Replicate | Concentration | (CFU/g) |
| 0-hours | BDL* | 6.2 × 10⁶ |
| 3 hours | | |
| Replicate 1 | 0.20% | 3.0 × 10⁷ |
| Replicate 2 | 0.17% | 2.9 × 10⁷ |
| Replicate 3 | 0.15% | 2.8 × 10⁷ |
| Average | 0.17% | 2.9 × 10⁷ |

*BDL = Below Detectable limits.

TABLE 2
Effect of incubation of chicken at 4° C. on biochemical and
microbiological parameters.

| | Carbon Dioxide | Bacterial Concentration |
|---|---|---|
| Replicate | Concentration | (CFU/g) |
| 0-hours | BDL* | 6.8 × 10⁴ |
| 48 hours | | |
| Replicate 1 | 1.0% | 4.3 × 10⁶ |
| Replicate 2 | 1.0% | 2.8 × 10⁶ |
| Replicate 3 | 0.6% | 4.2 × 10⁶ |
| Average | 0.87% | 3.8 × 10⁶ |
| Second batch of | | |
| chicken wings | | |
| 0-hours | BDL* | 7.8 × 10³ |
| 165 hours | | |
| Replicate 1 | 2.3% | 3.3 × 10⁷ |
| Replicate 2 | 3.5% | 4.4 × 10⁷ |
| Replicate 3 | 5.0% | 3.7 × 10⁷ |
| Average | 3.6% | 3.9 × 10⁷ |

*BDL = Below Detectable limits.
**NA = Not Applicable.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

That which is claimed is:

1. A device for detecting a presence of bacteria in a perishable food product comprising:
a gas-permeable sensor housing positionable within an interior of food packaging; and
a pH indicator positioned within the housing, for detecting a change in a gaseous bacterial metabolite concentration indicative of bacterial growth, a pH change effected by a presence of the metabolite, the housing and the pH indicator being safe for human consumption.

2. The device recited in claim 1, wherein the pH indicator is adapted to exhibit a radiative change selected from a group consisting of absorbance, fluorescence, and luminescence.

3. The device recited in claim 2, wherein the radiative change is detectable by at least one of visual means and an optical detection instrument.

4. The device recited in claim 2, wherein the pH indicator comprises means for undergoing a color change commensurate with a pH change.

5. The device recited in claim 4, further comprising a reference element positionable adjacent the color change undergoing means, the reference element having a substantially immutable color for use in comparing the color change undergoing means thereagainst.

6. The device recited in claim 5, wherein the color change undergoing means and the reference element are relatively positioned so that a color change undergone by the color change undergoing means forms a warning icon against the reference element.

7. The device recited in claim 1, wherein the housing is affixable to the food packaging interior by one of physical and chemical means.

8. The device recited in claim 1, wherein the pH indicator comprises an aqueous pH indicator and the housing comprises an at least partially transparent container for housing the pH indicator.

9. The device recited in claim 8, wherein the housing comprises one of a substantially transparent film and substantially transparent container, the housing gas permeable and charged-particle impermeable.

10. The device recited in claim 1, wherein the housing comprises a substantially transparent silicone, and the pH indicator comprises an aqueous pH indicator encapsulated within the silicone.

11. The device recited in claim 1, wherein the housing comprises a substantially transparent agar and the pH indicator comprises an aqueous pH indicator cured in a mixture with the agar.

12. The device recited in claim 11, wherein the housing further comprises a charged-particle-impermeable coating surrounding the agar-pH indicator mixture.

13. The device recited in claim 12, wherein the coating comprises one of a charged-particle-impermeable film and a silicone layer.

14. The device recited in claim 1, wherein the housing is positionable within the food packaging interior in spaced relation from the food product.

15. The device recited in claim 1, wherein the housing has a plurality of gas-permeable surfaces.

16. The device recited in claim 1, wherein at least a portion of the pH indicator is adapted to undergo a substantially irreversible change of state upon detecting the metabolite concentration change.

17. A device for detecting a presence of bacteria in a perishable food product comprising:
a gas-permeable sensor housing positionable within an interior of food packaging, the housing comprising a first container and a second container fluidically isolated therefrom;
means for establishing fluid communication between the first and the second container;
a pH indicator in a substantially desiccated state positioned within the first container, the pH indicator in a hydrated state adapted to detect a change in a gaseous bacterial metabolite concentration indicative of bacterial growth, a pH change effected by a presence of the metabolite; and
a hydrating solution positioned within the second container, wherein, in storage, the first and the second containers are fluidically isolated from each other, and, in use, the establishing means is actuated to rehydrate the pH indicator.

18. The device recited in claim 17, wherein the hydrating solution has sufficient alkalinity that a mixture of the pH indicator therewith results in an aqueous pH indicator having an initial pH in the alkaline range.

19. The device recited in claim 17, further comprising a container support and a fluid tube affixed to the support, and wherein the first and the second containers comprise a first and a second blister affixed to the support and in fluid communication with the tube, the establishing means comprises a frangible barrier positioned to block fluid access through the tube, a breaking of the frangible barrier establishing fluid communication between the first and the second blister.

20. A device for detecting a presence of bacteria in a perishable food product comprising:
a gas-permeable sensor housing positionable within an interior of food packaging, the housing comprising a first container and a second container fluidically isolated therefrom;
means for establishing fluid communication between the first and the second container;
a pH indicator in an acidic state positioned within the first container, the pH indicator in an alkaline state adapted to detect an increase in a gaseous bacterial metabolite concentration indicative of bacterial growth, a pH decrease effected by a presence of the metabolite; and
an alkaline solution positioned within the second container, wherein, in storage, the first and the second containers are fluidically isolated from each other, and, in use, the establishing means is actuated to raise the pH of the pH indicator into an alkaline range.

21. The device recited in claim 20, further comprising a container support and a fluid tube affixed to the support, and wherein the first and the second containers comprise a first and a second blister affixed to the support and in fluid communication with the tube, the establishing means comprises a frangible barrier positioned to block fluid access through the tube, a breaking of the frangible barrier establishing fluid communication between the first and the second blister.

22. A device for detecting a presence of bacteria in a perishable food product comprising:
a sealed sensor housing comprising a base material having a first pH in an alkaline range, the housing containing a gas for lowering the pH to a second pH during storage, the housing positionable within an interior of food packaging;
means for unsealing the housing preparatory to device usage, for releasing at least a portion of the gas and thereby raising the pH from the second pH to a third pH approximately equal to the first pH; and
a pH indicator positioned within the housing, for detecting a change in a gaseous bacterial metabolite concentration indicative of bacterial growth, a pH change effected by a presence of the metabolite, the pH indicator having a greater stability at the second pH than at the first pH.

23. The device recited in claim 22, wherein the base material comprises one of agar and silicone, the base material prepared with an alkaline solution.

24. The device recited in claim 22, wherein the gas comprises carbon dioxide.

25. A device for detecting a presence of bacteria in a perishable food product comprising:
a pH indicator for detecting a change in a gaseous bacterial metabolite concentration indicative of bacterial growth, a pH change effected by a presence of the metabolite;
a gas-permeable sensor housing adapted to contain the pH indicator, the housing having means for inhibiting light degradation of the pH indicator, the housing positionable within an interior of food packaging.

26. The device recited in claim 25, wherein the light degradation inhibiting means comprises a dye impregnated into the housing, the dye adapted to shield the pH indicator from at least ultraviolet wavelengths.

27. A device for detecting a presence of bacteria and lack of freshness in a perishable food product comprising:
a gas-permeable sensor housing positionable within an interior of food packaging;
a pH indicator positioned within the housing, for detecting a change in a gaseous bacterial metabolite concentration indicative of bacterial growth, a pH change effected by a presence of the metabolite; and
a time-temperature indicator positioned within the housing, for providing an integrated temperature history experienced by the food packaging; wherein
the pH indicator and the time-temperature indicator each contributes to a unitary calorimetric change.

28. The device recited in claim 27, further comprising a hydrating solution wherein:
the housing comprises a first container, a second container fluidically isolated therefrom, and means for establishing fluid communication therebetween;
the pH and the time-temperature indicators in storage reside in the first container in a substantially desiccated state, the pH indicator in a hydrated state adapted to detect the metabolite concentration change, the time-temperature indicator in a hydrated state adapted to provide the integrated temperature history;
the hydrating solution in storage resides in the second container; and
the establishing means serves to rehydrate and activate the pH and the time-temperature indicators when desired.

29. A device for detecting a presence of bacteria in a perishable food product comprising:
a gas-permeable sensor housing positionable within an interior of food packaging; and
an aqueous pH indicator positioned within the housing, for detecting a change in a gaseous volatile organic compound concentration indicative of bacterial growth, the gaseous volatile organic compound when exposed to an aqueous solution undergoing a reaction culminating in an increase in pH, the indicator having an initial pH in an acid range.

30. The device recited in claim 29, wherein the housing comprises one of silicone and a combination of agar and silicone.

31. The device recited in claim 30, wherein the indicator comprises an acid for establishing the initial pH.

32. The device recited in claim 29, wherein the indicator comprises one of bromothymol blue, phenol red, and cresol red, the indicator having an initial color indicating the acidic initial pH, the indicator turning a second color upon experiencing an increase in pH.

33. A device for detecting a presence of bacteria in a perishable food product comprising:
a gas-permeable sensor housing positionable within an interior of food packaging; and
a carbon dioxide indicator positioned within the housing, for detecting bacterial growth, the indicator comprising an aqueous solution including calcium hydroxide, an infusion of carbon dioxide into the housing effecting a detectable calcium carbonate precipitate.

34. A package for storing a perishable food product therein comprising:
a food product support;
a sealant positioned in substantially gas-impermeable sealing relation to the food support, thereby forming an interior space into which the food product may be packaged;
a gas-permeable sensor housing positionable within the interior space; and
a pH indicator positioned within the housing, for detecting a change in a gaseous bacterial metabolite concentration indicative of bacterial growth, a pH change effected by a presence of the metabolite.

35. The package recited in claim 34, wherein the pH indicator is adapted to exhibit a radiative change selected from a group consisting of absorbance, fluorescence, and luminescence.

36. The package recited in claim 35, wherein the radiative change is detectable by at least one of visual means and an optical detection instrument.

37. The package recited in claim 35, wherein the pH indicator comprises means for undergoing a color change commensurate with a pH change.

38. The package recited in claim 37, further comprising a reference element positionable adjacent the color change undergoing means, the reference element having a substantially immutable color for use in comparing the color change undergoing means thereagainst.

39. The package recited in claim 38, wherein the color change undergoing means and the reference element are relatively positioned so that a color change undergone by the color change undergoing means forms a warning icon against the reference element.

40. The package recited in claim 34, wherein the housing is affixable to the food packaging interior by one of physical and chemical means.

41. The package recited in claim 34, wherein the pH indicator comprises an aqueous pH indicator having an initial pH in an alkaline range.

42. The package recited in claim 34, wherein the housing comprises a substantially transparent silicone, and the pH indicator comprises an aqueous pH indicator encapsulated within the silicone.

43. The package recited in claim 34, wherein the housing comprises one of a substantially transparent film and substantially transparent container, the housing gas permeable and charged-particle impermeable.

44. The package recited in claim 34, wherein the housing comprises a substantially transparent agar and the pH indicator comprises an aqueous pH indicator cured in a mixture with the agar.

45. The package recited in claim 44, wherein the housing further comprises a charged-particle-impermeable coating surrounding the agar-pH indicator mixture.

46. The package recited in claim 45, wherein the coating comprises one of a charged-particle-impermeable film and silicone.

47. A method of detecting a presence of bacteria in a perishable food product comprising the steps of:
supporting a food product by a food packaging element;
sealing the food product and a gas-permeable sensor within the food packaging, the sensor comprising a pH indicator adapted to detect a change in a gaseous bacterial metabolite concentration indicative of bacterial growth, a pH change effected by a presence of the metabolite; and
monitoring the pH indicator for a bacterial concentration in the food product in excess of a predetermined level.

48. A method of packaging a perishable food product comprising the steps of:
supporting a food product by a food packaging element;
positioning a gas-permeable sensor housing within an interior of the food packaging element, the sensor comprising a pH indicator adapted to detect a change in a gaseous bacterial metabolite concentration indicative of bacterial growth, a pH change effected by a presence of the metabolite; and
sealing the food product and the housing within the food packaging.

49. The method recited in claim 48, wherein the sensor-positioning step comprises positioning the sensor in spaced relation from the food product.

50. A method of making a device for detecting a presence of bacteria in a perishable food product comprising the step of:
positioning a pH indicator within a gas-permeable sensor housing, the housing positionable within an interior of food packaging, the pH indicator adapted to detect a change in a gaseous bacterial metabolite concentration indicative of bacterial growth, a pH change effected by a presence of the metabolite, the housing and the pH indicator being safe for human consumption.

51. The method recited in claim 50, wherein the housing has a plurality of gas-permeable surfaces.

52. The method recited in claim 50, wherein at least a portion of the pH indicator is adapted to undergo a substantially irreversible change of state upon detecting the metabolite concentration change.

53. A method of making a device for detecting a presence of bacteria in a perishable food product comprising the steps of:
positioning a pH indicator in a substantially desiccated state within a first container, the pH indicator in a hydrated state adapted to detect a change in a gaseous bacterial metabolite concentration indicative of bacterial growth, a pH change effected by a presence of the metabolite a gas-permeable sensor housing positionable within an interior of food packaging, the housing comprising a first container and a second container fluidically isolated therefrom;
positioning a hydrating solution within a second container, the second container fluidically isolated from the first container;
providing means for establishing fluid communication between the first and the second container for use to rehydrate the pH indicator with the hydrating solution.

54. A method of making a device for detecting a presence of bacteria in a perishable food product comprising the steps of:
positioning a pH indicator in an acidic state within a first container, the pH indicator in an acidic state adapted to detect an increase in a gaseous bacterial metabolite concentration indicative of bacterial growth, a pH decrease effected by a presence of the metabolite second container fluidically isolated therefrom;
positioning an alkaline solution within a second container, the second container fluidically isolated from the first container;
providing means for establishing fluid communication between the first and the second container for use to raise a pH of the pH indicator with the alkaline solution.

55. A method of making a device for detecting a presence of bacteria in a perishable food product comprising the steps of:
positioning a pH indicator within a gas-permeable sensor housing, the housing positionable within an interior of food packaging, the pH indicator adapted to detect a change in a gaseous bacterial metabolite concentration indicative of bacterial growth;
adding a composition to at least one of the housing and the pH indicator, the composition having means for inhibiting light degradation of the pH indicator.

56. A method of making a device for detecting a presence of bacteria in a perishable food product comprising the steps of:
positioning a pH indicator within a gas-permeable sensor housing, the housing positionable within an interior of food packaging, the pH indicator adapted to detect a change in a gaseous bacterial metabolite concentration indicative of bacterial growth, a pH change effected by a presence of the metabolite;
positioning a time-temperature indicator within the housing, for providing an integrated temperature history experienced by the food packaging; wherein
the pH indicator and the time-temperature indicator each contributes to a unitary colorimetric change.

57. A method of making a device for detecting a presence of bacteria in a perishable food product comprising the step of:
positioning an aqueous pH indicator within a gas-permeable sensor housing, the indicator adapted to detect a change in a gaseous volatile organic compound concentration indicative of bacterial growth, the gaseous volatile organic compound when exposed to an aqueous solution undergoing a reaction culminating in an increase in pH, the indicator having an initial pH in an acid range.

58. A method of making a device for detecting a presence of bacteria in a perishable food product comprising the steps of:
positioning a gas-permeable sensor housing within an interior of food packaging; and
positioning a carbon dioxide indicator within a gas-permeable housing, the indicator comprising an aqueous solution including calcium hydroxide, an infusion of carbon dioxide into the housing effecting a detectable calcium carbonate precipitate.
